(19) United States
(12) Patent Application Publication
(10) Pub. No.: US 2004/0158164 A1
(43) Pub. Date: Aug. 12, 2004

(54) Method and system for detection of left ventricular hypertrophy

(76) Inventors: Patricia Arand, McMinnville, OR (US); Yoram Ariel, Portland, OR (US); Marco Dalla Gasperina, Vancouver, WA (US); Robert Warner, Tigard, OR (US)

Correspondence Address: PERKINS COIE LLP, PATENT-SEA, P.O. BOX 1247, SEATTLE, WA, 98111-1247, US

(21) Appl. No.: 10/730,695

(22) Filed: Dec. 8, 2003

Related U.S. Application Data
(63) Continuation-in-part of application No. 10/389,282, filed on Mar. 14, 2003, now abandoned.
(60) Provisional application No. 60/373,799, filed on Apr. 19, 2002.
(60) Provisional application No. 60/364,770, filed on Mar. 14, 2002.

(51) Int. Cl. A61B 5/04

(52) U.S. Cl. 600/509

(57) ABSTRACT

A method and system for diagnosing left ventricular hypertrophy (“LVH”) based on ECG data. An LVH system provides an indication of the likelihood that a patient has LVH based on an analysis of the patient's ECG data. The LVH system estimates the left ventricular mass index (“LVMI”) for the patient based on gender-specific formulae that use time-voltage data derived from the ECG data. After the LVH system calculates the estimated LVMI for the patient, it then classifies the estimated LVMI to indicate the likelihood that the patient has LVH. The LVH system may then select various statements of condition and rationale based on the patient's classification.

### CROSS-REFERENCE TO RELATED APPLICATIONS

This application is a continuation-in-part of U.S. patent application Ser. No. 10/389,282 entitled “METHOD AND SYSTEM FOR DETECTION OF LEFT VENTRICULAR HYPERTROPHY” filed Mar. 14, 2003, which application claims the benefit of U.S. Provisional Application No. 60/373,799 entitled “IMPROVED ELECTROCARDIOGRAPHIC DETECTION OF LEFT VENTRICULAR HYPERTROPHY,” filed Apr. 19, 2002, and U.S. Provisional Application No. 60/364,770 entitled “ECG/SOUND ALGORITHM ADAPTER,” filed Mar. 14, 2002, both of which are incorporated herein by reference in their entireties.

### TECHNICAL FIELD

The described technology relates to a computer system for evaluating the presence of left ventricular hypertrophy in a patient.

### BACKGROUND

The electrocardiogram (“ECG”) is an important tool for monitoring heart activity and diagnosing heart conditions. The ECG is a recording of the electrical activity of the heart. This electrical activity causes the heart to contract. The contraction in turn causes blood to be pumped throughout the body. This electrical activity is spontaneously generated. As the cells within the heart change from a negative potential to a positive potential (depolarization), the muscles within the heart contract. Conversely, when the cells change from a positive to a negative potential (repolarization), the muscles return to their noncontracted state. The periodic contraction of the heart causes the pumping action. This spontaneous electrical activity typically occurs about once a second. By analyzing a patient's ECG, various cardiac abnormalities, such as ischemia, can be detected.

The ECG has been used to diagnose left ventricular hypertrophy (“LVH”). Progressive LVH often results in congestive cardiac failure and serious arrhythmias, and as a result, it is a major cause of death and disability throughout the world. Detecting LVH is important because the available therapeutic interventions for LVH per se and for three of its common causes—coronary artery disease, hypertension and valvular heart disease—have become increasingly effective. Therefore, identifying patients who have begun to develop LVH can favorably affect their prognoses. The ubiquity, ease of acquisition, and low cost of the ECG makes it an especially useful test for identifying and following patients with LVH. Furthermore, the ECG can detect important concurrent conditions (e.g., arrhythmias and evidence of myocardial infarction) that are both often associated with LVH and that commonly influence physicians' decisions regarding the management of their patients. Unfortunately, the poor diagnostic performance of traditional ECG criteria and of previously developed ECG computerized algorithms limits the utility of the ECG as a reliable screening test for LVH. It would be desirable to have an improved system for diagnosing LVH based on an ECG.

### BRIEF DESCRIPTION OF THE DRAWINGS

FIG. 1 is an ECG illustrating results of the LVH system in one embodiment.

FIG. 2 is a diagram illustrating the position of leads V1-V6 and an ECG complex.

FIG. 3 is a block diagram illustrating the components of the LVH system in one embodiment.

FIG. 4 is a flow diagram illustrating the processing of the analyze ECG for LVH component in one embodiment.

FIG. 5 is a flow diagram illustrating the processing of the generate initial classification component in one embodiment.

FIG. 6 is a flow diagram illustrating the processing of the adjust classification component in one embodiment.

FIG. 7 is a flow diagram illustrating the processing of the adjust component in one embodiment.

FIG. 8 is a flow diagram illustrating the processing of the select LVH statement component in one embodiment.

FIG. 9 is a flow diagram illustrating the processing of the select rhythm interpretation statement component in one embodiment.

FIG. 10 is a flow diagram illustrating the processing of the select rationale statement component in one embodiment.

FIG. 11 is a flow diagram illustrating the processing of the generate evidence graph component in one embodiment.

### DETAILED DESCRIPTION

A method and system for diagnosing left ventricular hypertrophy (“LVH”) based on electrocardiogram (“ECG”) data is provided. An LVH system in one embodiment provides an indication of the likelihood that a patient has LVH based on gender and an analysis of the patient's ECG data. The LVH system initially determines whether the patient's condition is such that the LVH system cannot reliably assess the likelihood that the patient has LVH. For example, if the patient has a right bundle branch block, the results of the LVH system may not be reliable. The LVH system then estimates the left ventricular mass index (“LVMI”) for the patient based on gender-specific formulae that use time-voltage data derived from the ECG data. The gender-specific formulas combine parameters representing the area of the ST segment (from lead V1 for males and lead I for females), the area of the initial STT segment (from lead V5 for males and lead V6 for females), the QRS time integral of the Frank XZ vector, and a modified Cornell Product for the patient. The LVH system combines these parameters using gender-specific weighting factors. These specific parameters and weighting factors can be derived from a collection of ECG data using various data modeling techniques such as a least squares linear regression analysis. Suitable data modeling techniques are described in William H. Press, et al., Numerical Recipes in C++: The Art of Scientific Computing, (2d ed. Cambridge Univ. Press 1992, reprint 1999), which is hereby incorporated by reference. In one embodiment, a gender-specific formula can be generated using ECG data for a patient population and each patient's measured LVM. The LVM of a patient can be measured using various well-known techniques, such as using an echocardiograph to measure left ventricular mass. LVMI is calculated by dividing LVM by an estimate of the patient's body surface area. Data modeling can then be used to identify the gender-specific parameters and weighting factors based on how well the ECG data and the LVMI measurements represent data of a more general population. For example, more parameters (i.e., variables) can be used when modeling if the data is highly representative of the more general population.

After the LVH system calculates the estimated LVMI for the patient, it then classifies the estimated LVMI to indicate the likelihood that the patient has LVH. The LVH system performs this classification on a gender-specific basis. In one embodiment, a classification ranges from 0 to 3, where 0 indicates no evidence of LVH and 3 indicates the strongest evidence of LVH. The LVH system then adjusts the patient's classification based on various confounders. For example, the LVH system may reduce the patient's classification if the patient has a left anterior fascicular block. The adjustments may be gender-specific. The LVH system may then select various statements of condition and statements of rationale based on the patient's classification and parameters.

Identify Excluders and Confounders

The LVH system in one embodiment identifies excluder conditions and confounder conditions based on a rhythm interpretation and ECG measurement analysis. If an excluder condition is identified, then the LVH system does not perform its LVH detection analysis because the excluder condition may cause the results to be unreliable. The LVH system, however, adjusts its LVH detection analysis to account for the identified confounder conditions. The following Excluder/Confounder table lists excluder and confounder conditions used by the LVH system in one embodiment.

Excluder/Confounder Table

| Condition | LVH |
|---|---|
| Age less than 18 | Excluder |
| Right Bundle Branch Block (RBBB) | Excluder |
| Complete Left Bundle Branch Block (CLBBB) | Excluder |
| Wolff Parkinson White Syndrome (WPW) | Excluder |
| Paced or Ventricular Rhythm (VP, VR) | Excluder |
| Ventricular Tachycardia (VT) | Excluder |
| Dextrocardia | Excluder |
| QRS Duration > 140 milliseconds | Excluder |
| Intraventricular Conduction Delay (IVCD) | Confounder |
| Intraventricular Conduction Block (IVCB) | Confounder |
| Left Anterior Fascicular Block (LAFB) | Confounder |

Estimate LVMI

The LVH system in one embodiment estimates the LVMI using gender-specific formulae. The formulae and weighting factors for males are as follows:

$$\begin{matrix}LVMI = \frac{1}{1000} [ {a}_{1} ASTV1 + \\ {a}_{2} ASTTV5 + {a}_{3} Axz + {a}_{4} CPV3 + {b}_{1} ]\end{matrix} \qquad ( male formula )$$

a₁=0.901

a₂=−5.812

a₃=0.403 (male factors)

a₄=0.085

b₁=67439

where the parameter ASTV1 is the area of the ST segment, defined from the J point extending 80 ms, as measured by lead V1; the parameter ASTTV5 is the area of a portion of the STT segment, defined from the J point extending 176 ms, as measured on lead V5; the parameter Axz is the QRS time integral of the derived Frank vector amplitude in the XZ plane; and CPV3 is a modified Cornell Product that is the Cornell Voltage times the QRS time duration in lead V3. The Cornell Voltage is the maximum voltage of the R wave as measured on lead aVL (i.e., RaVL) plus the maximum voltage of the S wave as measured on lead V3 (i.e., SV3). Each parameter represents a time-voltage unit of measurement.

The formula and weighting factor for females are as follows:

$$\begin{matrix}LVMI = \frac{1}{1000} [ {a}_{1} ASTI + \\ {a}_{2} ASTTV6 + {a}_{3} Axz + {a}_{4} CPV3 + {b}_{1} ]\end{matrix} \qquad ( female formula )$$

a₁=−1.55

a₂=−1.66

a₃=0.285 (female factors)

a₄=0.109

b₁=62041

where the parameters Axz and CPV3 are the same as for males. The parameter ASTI is the area of the ST segment as measured in lead I. The parameter ASTTV6 is a portion of the area of the STT segment, defined from the J point extending 240 ms, as measured on lead V6.

In one embodiment, these formulae and factors are derived using linear regression techniques applied to a population with known ECG data and LVMI measurements. A regression equation is a mathematical equation that can be used to predict the values of one dependent variable (e.g., LVMI) from known values of one or more independent variables (e.g., ECG measurements). Linear regression attempts to model the relationship between the dependent and independent variables by fitting a linear equation to observed data (the dependent variable). A linear regression is based upon an equation of the form Y=a1*x1+a2*x2+ . . . +an*xn+b, where x is an independent variable and Y is the dependent variable.

One skilled in the art will appreciate that before attempting to fit a linear regression model to the observed data, it should be determined whether there is a relationship between the variables of interest. The knowledge of this relationship can be used to help select the independent variable that can best model the dependent variable. When the linear regression model uses several independent variables, the particular combination of independent variables chosen and the combining coefficients determine how good the model predicts the dependent variable. In the case of ECG data, there are many candidate independent variables. One skilled in the art will appreciate that an analysis of the separate power of independent variables to predict the dependent variable along with their power in combination is needed to determine the best model for prediction given the overall goals (e.g., accuracy) and constraints (e.g., speed) of the model.

Generate Initial Classification

The LVH system classifies the estimated LVMI to indicate the likelihood that the patient has LVH. In one embodiment, the classification value can be 0 to 3. A classification of 0 indicates no evidence of LVH, and a classification of 3 indicates strong evidence of LVH. Thus, the same classification value for male or female is intended to represent the same likelihood that the patient has LVH. The following table lists LVMI conditions and the corresponding classification for males. Each row of the table specifies a classification rule.

Initial Classification Table (males)

| Condition | Classification |
|---|---|
| LVMI <= 110 | 0 |
| 110 < LVMI <= 120 | 1 |
| 120 < LVMI <= 130 | 2 |
| 130 < LVMI | 3 |

The following table lists LVMI conditions and the corresponding classification values for females.

Initial Classification Table (females)

| Condition | Classification |
|---|---|
| LVMI <= 93 | 0 |
| 93 < LVMI <= 100 | 1 |
| 100 < LVMI <= 110 | 2 |
| 110 < LVMI | 3 |

Adjust Classification for Confounders/Parameters

When the initial classification is not zero, the LVH system adjusts the classification to account for gender-specific confounders and patient-specific parameters. In one embodiment, three separate adjustments are performed for males and two for females. The LVH system determines which confounder/parameter conditions are satisfied and adjusts the classification as indicated. The first adjustment is based primarily on conduction blocks and high QRS voltages (“HQV”). The following table lists the rules (i.e., conditions and adjustments) for males for the first adjustment.

Block/HQV Table (males)

| Condition | Adjustment |
|---|---|
| Class > 2 and | Class = Class − 1 |
| BLOCK and | |
| HQV | |
| Class > 2 and | Class = 1 |
| BLOCK and | |
| not HQV | |
| Class > 2 and not BLOCK and | Class = Class − 1 |
| QRSduration > 100 and | |
| 0.000085*CPV3/LVMI > 0.33 and | |
| not HQV | |

where HQV is true when high QRS voltages are present and BLOCK is true when conduction blocks that are confounders, but not excluders, are present. HQV can be set to true based on the Sokolow-Lyon voltages (i.e., SV1+max (RV5, RV6) or RaVL) or other ECG criteria. Examples of conduction blocks include a rhythm interpretation of left anterior fascicular block (“LAFB”) and of an intraventricular conduction delay (“IVCD”) with QRS duration less than or equal to 140 milliseconds.

The following table lists the rules for females for the first adjustment.

Block/HQV Table (females)

| Condition | Adjustment |
|---|---|
| Class ≧ 2 and | Class = Class − 1 |
| BLOCK and | |
| HQV | |
| Class ≧ 2 and | Class = 1 |
| BLOCK and | |
| not HQV | |
| Class ≧ 2 and | Class = Class − 1 |
| not BLOCK and QRSduration > 90 | |
| and 0.000109*CPV3/LVMI > 0.20 and | |
| not HQV | |

The second adjustment is made only for males and relates to the area of the T wave. The following table lists the rules for males. The rules in this table are applied serially.

ATP4 Table (males)

| Condition | Adjustment |
|---|---|
| Class >= 1 and | Class = Class − 1 |
| ATPV4 > 150*HTMagnitude | |

where ATPV4 is the positive area of the T wave as measured by lead V4 and HTMagnitude is the T amplitude of the horizontal (x,y) vector.

The third adjustment relates to the patient's parameters used to estimate the LVMI. The following table lists the rule for males for the third adjustment.

Parameter Table (males)

| Condition | Adjustment |
|---|---|
| Axz <= 65000 and | Class = 0 |
| CPV3 <= 200000 and | |
| ASTTV5 >= −500 and | |
| ASTV1 <= 6000 | |

The following table lists the rule for females for the third adjustment.

Parameter Table (females)

| Condition | Adjustment |
|---|---|
| Axz <= 60000 and | Class = 0 |
| CPV3 <= 150000 and | |
| ASTTV6 >= −1000 and | |
| ASTI <= −2000 | |

Select LVH Statements

The LVH system selects an LVH statement based on the patient's classification. The LVH statements are the same for both genders. The LVH statement is selected based on the classification as indicated by the following table.

LVH Statements Table

| Condition | LVH Statements |
|---|---|
| Class = 0 | (no statement) |
| Class = 1 | “MINIMAL EVIDENCE FOR LVH” |
| Class = 2 | “MODERATE EVIDENCE FOR LVH” |
| Class = 3 | “STRONG EVIDENCE FOR LVH” |

The LVH system selects additional statements based on traditional ECG system for HQV according to the following table.

HQV Statements Table

| Condition | Statement |
|---|---|
| Class = 0 and no HQV | (no statement) |
| Class = 0 and HQV | “HIGH QRS VOLTAGES IN ONE OF |
| | R(aVL), S(V1), R(V5), |
| | R(V5/V6) + S(V1) − |
| | MAYBE NORMAL VARIANT” |
| Class >= 1 and HQV | “HIGH QRS VOLTAGES IN ONE OF |
| | R(aVL), S(V1), R(V5), |
| | R(V5/V6) + S(V1)” |

Select LVH Rationale Statements

The LVH system selects rationale statements based on the gender-specific parameters generated for the estimation of LVMI. The following table lists the rules for rationale statements for males.

Rationale Statements Table (males)

| Condition | Statements |
|---|---|
| Axz > 65000 and not HQV | “INCREASED QRS AREA” |
| CPV3 > 200000 | “INCREASED [aVL(R) + V3(S)]*QRS |
| | DURATION PRODUCT” |
| ASTTV5 <− 500 and | “STT ABN IN V5” |
| ASTV1 <= 6000 | |
| ASTTV5 >= −500 and | “STT ABN IN V1” |
| ASTV1 > 6000 | |
| ASTTV5 <− 500 and | “STT ABN IN V1/V5” |
| ASTV1 > 6000 | |

The following table lists the rules for rationale statements for females.

Rationale Statements Table (females)

| Condition | Statements |
|---|---|
| Axz > 60000 and not HQV | “INCREASED QRS AREA” |
| CPV3 > 150000 | “INCREASED [aVL(R) + V3(S)]*QRS |
| | DURATION PRODUCT” |
| ASTTV6 <− 1000 and | “STT ABN IN V6” |
| ASTI >= −2000 | |
| ASTTV6 >= −1000 and | “STT ABN IN I” |
| ASTI <− 2000 | |
| ASTTV6 <− 1000 and | “STT ABN IN I/V6” |
| ASTI <− 2000 | |

Implementation Details

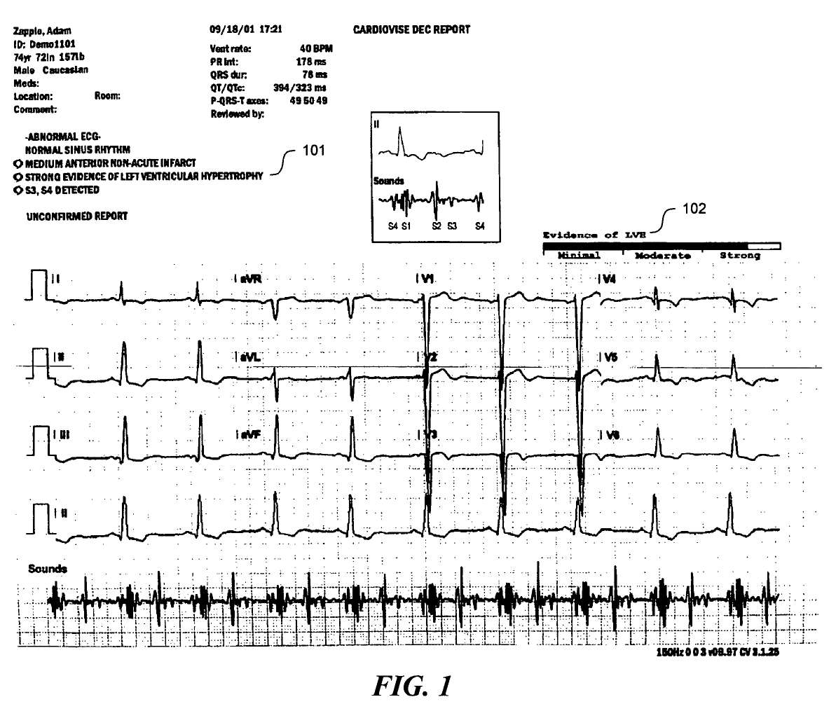

FIG. 1 is an ECG report illustrating results of the LVH system in one embodiment. The LVH system outputs one or more statements relating to the detection of LVH. In this example, the LVH system outputs the statement “Strong Evidence of Left Ventricular Hypertrophy” 101 selected from the LVH statements table. The LVH system also outputs an evidence graph 102. The evidence graph rates the evidence of LVH as minimal, moderate, or strong. In this example, the evidence of LVH is relatively strong as indicated by the graph. If the LVH system could not evaluate the LVH because of an excluder, the LVH system would indicate that the LVH cannot be evaluated and output a description of the excluder selected from the Excluder/Confounder table.

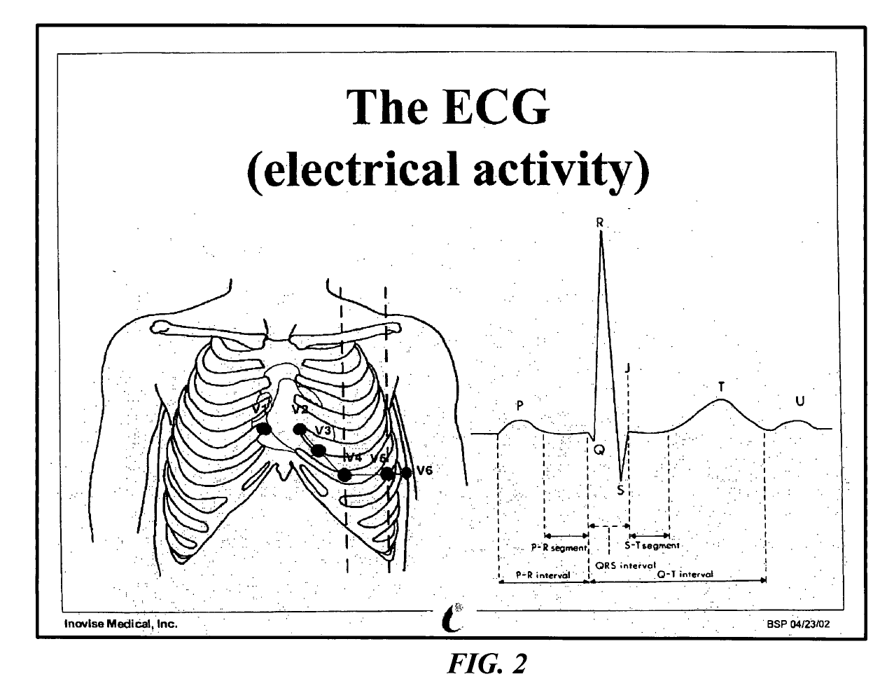

FIG. 2 is a diagram illustrating the position of leads V1-V6 and an ECG complex. The ECG complex illustrates various measurements used by the LVH system. The measurements include the QRS interval, the J point, the ST segment, and so on.

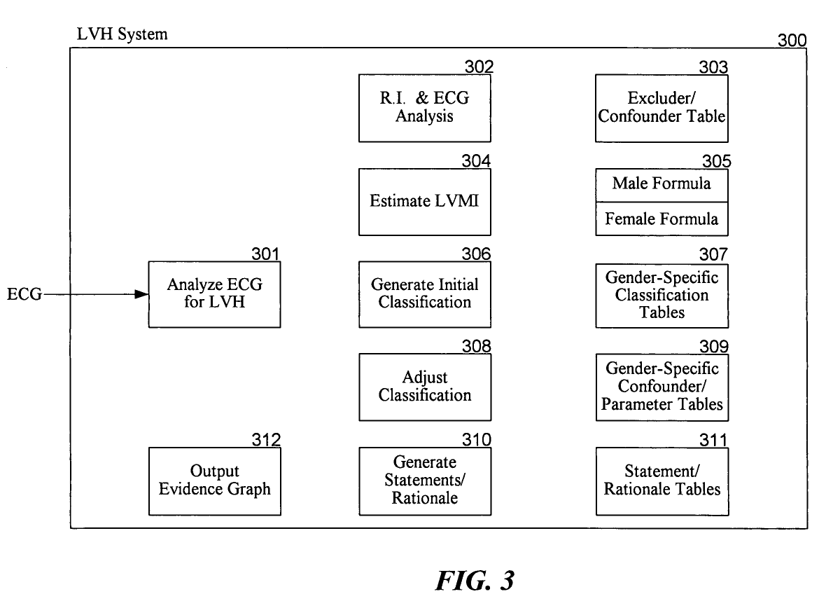

FIG. 3 is a block diagram illustrating the components of the LVH system in one embodiment. The LVH system 300 includes an analyze ECG for LVH component 301 that inputs a patient's ECG and invokes various components to identify evidence of LVH in the patient. The components include a rhythm interpretation and ECG measurement analysis component 302, an estimate LVMI component 304, a generate initial classification component 306, an adjust classification component 308, a generate statements/rationale component 310, and an output evidence graph component 312. The rhythm interpretation and ECG measurement analysis component determines whether any excluders or confounders exist as defined by the Excluder/Confounder table 303. The estimate LVMI component calculates the gender-specific parameters for the patient and then applies a gender-specific formula as defined by formula component 305 to estimate the LVMI for the patient. The generate initial classification component inputs the estimated LVMI and uses the rules in the gender-specific Initial Classification tables 307 to generate an initial classification for the patient. The adjust classification component uses the rules in the gender-specific confounder/parameter tables 309 to adjust the patient's classification. The generate statements/rationale component uses the patient's classification and parameters to select the applicable statements from the statements/rationale tables 311.

The LVH system may be implemented on a computer that includes a central processing unit, memory, input devices (e.g., keyboard and pointing devices), output devices (e.g., display devices), and storage devices (e.g., disk drives). The memory and storage devices are computer-readable media that may contain instructions that implement the LVH system. The LVH system may be a stand-alone system or may be integrated into a conventional ECG monitoring system.

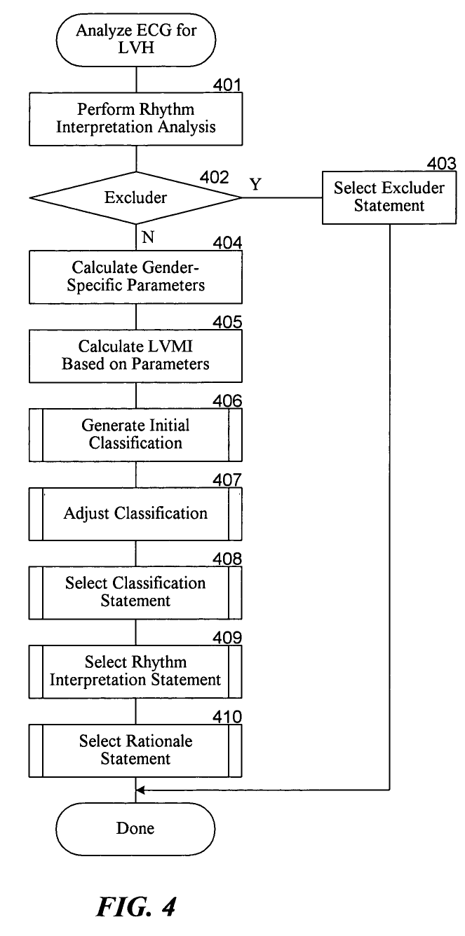

FIGS. 4-11 are flow diagrams illustrating the processing of the components of the LVH system in one embodiment. One skilled in the art will appreciate that the flow diagrams describe the overall function of the components and the actual implementation of a component can vary considerably based on various design goals, such as cost and speed. One skilled in the art will also appreciate that the functions of the LVH system can be grouped into more or fewer components and additional components can be defined.

FIG. 4 is a flow diagram illustrating the processing of the analyze ECG for LVH component in one embodiment. The component inputs ECG data for a patient and controls the invocation of the other components of the LVH system. In block 401, the component performs the rhythm interpretation and ECG measurement analysis. In one embodiment, this analysis may be provided by the ECG monitoring system in which the LVH system is embedded. In decision block 402, the component determines whether the patient has any of the excluder conditions as indicated by the Excluder/Confounder table. If so, the component selects the appropriate excluder statements in block 403 and then completes, else the component continues at block 404. In block 404, the component calculates the gender-specific parameters for use in estimating the LVMI according to the gender-specific formulae. In block 405, the component estimates the LVMI using the gender-specific formula and the gender-specific parameters. In block 406, the component invokes the generate initial classification component to provide an initial classification for the patient in a gender-specific way. In block 407, the component invokes the adjust classification component to adjust the classification in a gender-specific way based on confounders and the patient's parameters. In block 408, the component invokes the select classification statement component to select the appropriate statement from the LVH Statements table. In block 409, the component invokes the select rhythm interpretation and ECG measurements statement component to select the appropriate statement from the Rhythm Interpretation Statements table. In block 410, the component invokes the select rationale statement component to select a rationale for the LVH analysis from the Rationale Statements tables.

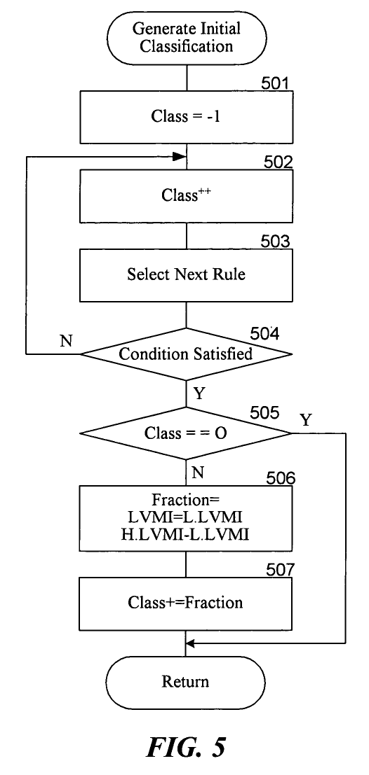

FIG. 5 is a flow diagram illustrating the processing of the generate initial classification component in one embodiment. The component is passed the estimated LVMI along with the patient's gender. The component uses the Initial Classification table of the appropriate gender to determine the classification. In block 501, the component initializes the classification. In blocks 502-504, the component loops selecting the rules of the Initial Classification table until a condition of the rule is satisfied. In block 502, the component increments the classification, initially setting it to zero. In block 503, the component selects the next rule from the Initial Classification table of the appropriate gender. In decision block 504, if the condition of the selected rule is satisfied, then the component continues at block 505, else the component loops to block 502 to select the next rule. In decision block 505, if the value of the current classification is zero, then the component returns, else the component continues at block 506. In block 506, the component calculates a fraction for the classification. The fraction in one embodiment used for graphical representation is defined as the estimated LVMI minus the low LVMI of the condition divided by the high LVMI minus the low LVMI of the condition. For example, if the LVMI for a male is 133, then the fraction is 0.33 (i.e., (133−130)/(139−130)). In block 507, the component adds the fraction to the classification and then returns.

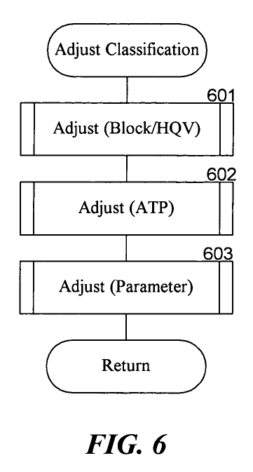

FIG. 6 is a flow diagram illustrating the processing of the adjust classification component in one embodiment. The component is passed various ECG-based indicators for use in calculating the gender-specific confounder adjustments. In block 601, the component invokes an adjust component passing an indication of the Block/HQV table. The adjust component adjusts the current classification based on the passed table. In block 602, the component invokes the adjust component passing the ATPV4 table. In block 603, the component invokes the adjust component passing the Parameter table. The component then returns.

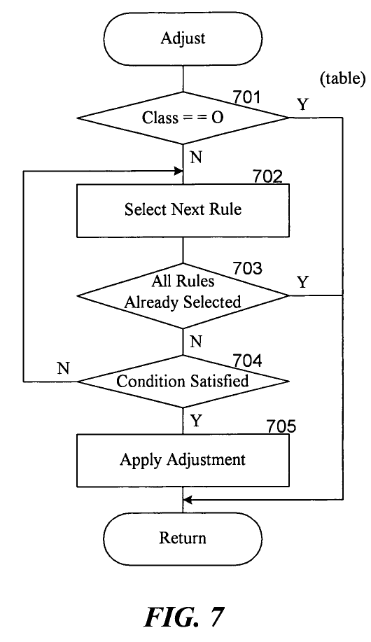

FIG. 7 is a flow diagram illustrating the processing of the adjust component in one embodiment. The component is passed a table and evaluates each of the conditions within the table to determine if it is satisfied. If satisfied, the component adjusts the classification as indicated. In decision block 701, if the classification is equal to zero, then the component returns, else the component continues at block 702. In blocks 702-704, the component loops selecting each rule until it locates a rule that is satisfied. In one embodiment, the LVH system processes all the rules of the ATPV4 table serially so that multiple rules can be applied. In block 702, the component selects the next rule of the passed table. In decision block 703, if all the rules of the passed table have already been selected, then the component returns, else the component continues at block 704. In decision block 704, if the condition of the selected rule is satisfied, then the component continues at block 705, else the component loops to block 702 to select the next rule. In block 705, the component applies the adjustment indicated by the selected rule and then returns.

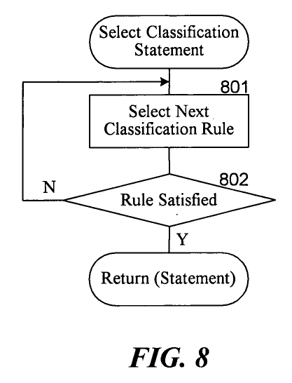

FIG. 8 is a flow diagram illustrating the processing of the select LVH statement component in one embodiment. The component is passed a classification and selects the appropriate LVH statement from the LVH Statements table. In block 801, the component selects the next rule from the LVH Statements table. In decision block 802, if the condition of the selected rule is satisfied, then the component returns the statement of the selected rule, else the component loops to block 801 to select the next rule.

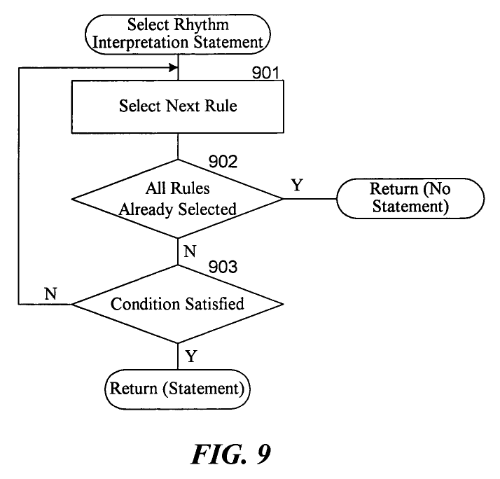

FIG. 9 is a flow diagram illustrating the processing of the select rhythm interpretation statement component in one embodiment. In blocks 901-903, the component loops determining which rhythm interpretation statement to select. In block 901, the component selects the next rule of the Rhythm Interpretation Statements table. In decision block 902, if all the rules have already been selected, then the component returns an indication of no rhythm interpretation statement, else the component continues at block 903. In decision block 903, if the condition of the selected rule is satisfied, then the component returns the statement associated with the selected rule, else the component loops to block 901 to select the next rule.

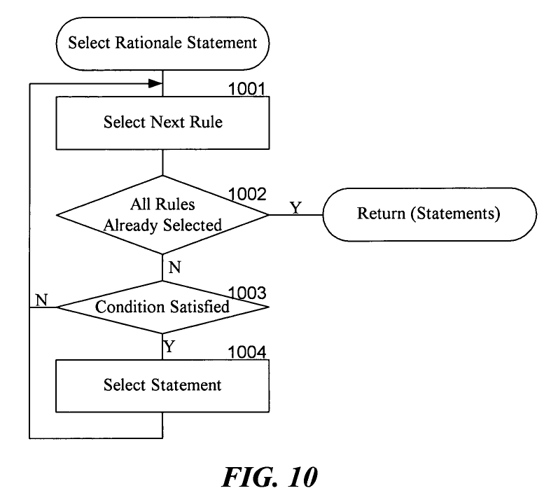

FIG. 10 is a flow diagram illustrating the processing of the select rationale statement component in one embodiment. The component selects the rationale statements that apply to this patient. In blocks 1001-1004, the component loops determining which rationale statements apply. In block 1001, the component selects the next rule of the Rationale Statements table. In decision block 1002, if all the rules of the Rationale Statements table have already been selected, then the component returns the statements that have been selected, else the component continues at block 1003. In decision block 1003, if the condition of the selected rule is satisfied, then the component continues at block 1004, else the component loops to block 1001 to select the next rule. In block 1004, the component selects the statement of the selected rule and then loops to block 1001 to select the next rule.

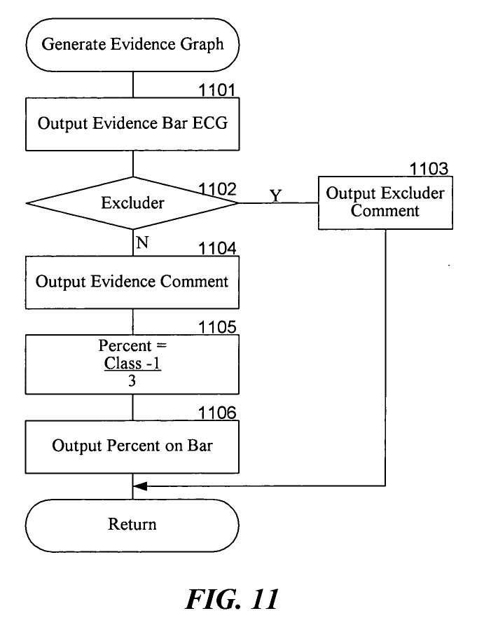

FIG. 11 is a flow diagram illustrating the processing of the generate evidence graph component in one embodiment. In block 1101, the component outputs the evidence ECG graph (e.g., a bar graph). In decision block 1102, if any excluder conditions were identified for this patient, then the component continues at block 1103, else the component continues at block 1104. In block 1103, the component outputs the appropriate excluder statement and then completes. In block 1104, the component outputs the selected LVH statement for this component. In block 1105, the component calculates the percent for the evidence graph to be filled in. In block 1106, the component fills in that percent on the evidence graph and then completes.

One skilled in the art will appreciate that although specific embodiments of the LVH system have been described herein for purposes of illustration, various modifications may be made without deviating from the spirit and scope of the invention. Accordingly, the invention is not limited except by the appended claims.

I/we claim:

1. A method in a computer for estimating an LVMI for a patient based on ECG data, the method comprising:
calculating an ST time-voltage parameter representing the area of the ST segment of the ECG data;
calculating an STT time-voltage parameter representing a portion of the area of the STT segment the ECG data;
calculating a QRS time-voltage parameter representing a QRS time integral;
calculating a modified Cornell Product time-voltage parameter;
adjusting each calculated time-voltage parameter based on a weighting factor for that parameter; and
summing the adjusted time-voltage parameters as an estimate of the LVMI.

2. The method of claim 1 wherein the weighting factors are gender-specific.

3. The method of claim 1 wherein the ST time-voltage parameter for a male is based on lead V1.

4. The method of claim 1 wherein the ST time-voltage parameter for a female is based on lead I.

5. The method of claim 1 wherein the STT time-voltage parameter is based on lead V5 for males.

6. The method of claim 1 wherein the STT time-voltage parameter is defined from the J point extending approximately 176 ms for males.

7. The method of claim 1 wherein the STT time-voltage parameter is based on lead V6 for females.

8. The method of claim 1 wherein the STT time-voltage parameter is defined from the J point extending approximately 240 ms for females.

9. The method of claim 1 wherein the QRS time-voltage parameter is a QRS time integral of a derived Frank vector amplitude in the XZ plane.

10. A method in a computer for diagnosing LVH in a patient, the method comprising:
estimating an LVMI based on time-voltage parameters derived from ECG data of the patient;
generating an initial likelihood of LVH based on the estimated LVMI; and
adjusting the initial likelihood based on one or more confounders that have been identified for the patient to give a likelihood that the patient has LVH.

11. The method of claim 10 wherein the time-voltage parameters include an ST time-voltage parameter, an STT time-voltage parameter, a QRS time-voltage parameter, and a modified Cornell Product time-voltage parameter.

12. The method of claim 11 including adjusting each calculated time-voltage parameter based on a weighting factor for that parameter.

13. The method of claim 12 including summing the adjusted time-voltage parameters as an estimate of the LVMI.

14. The method of claim 10 wherein the generating of the initial likelihood is gender-specific.

15. The method of claim 10 wherein the adjusting of the initial likelihood is gender-specific.

16. The method of claim 10 wherein the confounders include conduction blocks.

17. The method of claim 16 wherein the adjustment is based in part on whether a high QRS voltage is detected.

18. The method of claim 16 wherein the adjustment is based in part on the duration of the QRS.

19. The method of claim 10 including additionally adjusting the initial likelihood based on area of the T wave in V4.

20. The method of claim 10 including additionally setting the likelihood to negligible when the time-voltage parameters do not meet certain thresholds.

21. The method of claim 10 including selecting a statement of the diagnosis based on whether the adjusted likelihood indicates no, minimal, moderate, or strong evidence of LVH.

22. The method of claim 10 including selecting a rationale statement for the diagnosis based on time-voltage parameters.

23. The method of claim 22 wherein the selection of the rationale statement is gender-specific.

24. The method of claim 10 wherein the estimating of LVMI is gender-specific.

25. The method of claim 10 wherein the estimating is based on data modeling of ECG data and LVMI measurements for a population.

26. A method in a computer for estimating the LVMI for a patient, the method comprising:
calculating time-voltage parameters derived from ECG data of the patient;
adjusting the calculated time-voltage parameters based on a weighting factor for each parameter; and
combining the adjusted time-voltage parameter to estimate the LVMI for the patient
wherein the parameters and weight factors are derived from data modeling of ECG data and LVMI measurements for a population.

27. The method of claim 26 wherein the time-voltage parameters include an ST time-voltage parameter, an STT time-voltage parameter, a QRS time-voltage parameter, and a modified Cornell Product time-voltage parameter.

28. The method of claim 26 wherein the estimating is gender-specific.

29. The method of claim 26 including adjusting each calculated time-voltage parameter based on a weighting factor for that parameter.

30. The method of claim 26 wherein the weighting factors are gender-specific.

31. The method of claim 26 wherein the combining includes summing the adjusted time-voltage parameters.

32. A method in a computer system for presenting evidence of LVH in a patient, the method comprising:
providing an indication of the strength of the evidence that a patient has LVH; and
displaying a graph in which the strength of the evidence is indicated by a length of the graph.

33. The method of claim 32 wherein the graph is a horizontal bar graph.

34. The method of claim 32 wherein the bar graph is displayed on an ECG printout.

35. The method of claim 32 including displaying a statement of the evidence adjacent to the graph.

36. The method of claim 32 including displaying a statement of rationale adjacent to the graph.

37. The method of claim 32 wherein the graph is a bar graph and the evidence ranges from minimal to strong.

38. The method of claim 32 including suppressing the displaying of the graph when there is no evidence of LVH.

39. A computer system for diagnosing LVH in a patient, comprising:
means for modeling LVMI based on ECG data and LVMI measurements for a population;
means for estimating LVMI for a person based on the model and ECG data for the person; and
means for generating an indication of the likelihood of LVH based on the estimated LVMI and adjustment rules.

40. A computer system for assisting in the diagnosing of LVH in a patient, comprising:
a component that estimates an LVMI for the patient based on gender-specific, time-voltage parameters and ECG data of the patient; and
a component that generates a likelihood that the patient has LVH based on the estimated LVMI and various adjustment rules.

41. The computer system of claim 40 wherein the adjustment rules are based on the presence of confounders in the patient.

42. The computer system of claim 40 wherein the adjustment rules are based on threshold values for the time-voltage parameters.

43. The computer system of claim 40 including:
a component that selects statements of evidence of LVH based on the generated likelihood.
